(19) United States
(12) Patent Application Publication
(10) Pub. No.: US 2008/0020020 A1
(43) Pub. Date: Jan. 24, 2008

(54) Beverage

(75) Inventor: Ann Williamson, Cedex (FR)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco Inc, d/b/a UNILEVER

(21) Appl. No.: 11/880,791

(22) Filed: Jul. 24, 2007

(30) Foreign Application Priority Data
Jul. 24, 2006 (EP) EP06291197

(51) Int. Cl. A61K 47/00; A23F 3/00; A23F 3/40

(52) U.S. Cl. 424/439; 426/533; 426/548; 426/597

(57) ABSTRACT

The present invention provides a beverage having an energy content of less than 17 kJ per 100 g, wherein the beverage comprises catechins in amount (C) of from 0.04 to 0.5% by weight of the beverage, and non-nutritive sweetener in an amount satisfying the following condition:
34<S/C<115, wherein S is the sweetness of the non-nutritive sweetener in terms of sucrose.

### TECHNICAL FIELD OF THE INVENTION

The present invention relates to beverages. More particularly the invention relates to beverages containing catechins, such as tea-based beverages.

### BACKGROUND OF THE INVENTION

Green tea is a popular beverage which has been consumed in China and Japan for many hundreds of years. Recently, extensive laboratory research and epidemiologic studies have shown that compounds present in green tea (particularly catechins) may reduce the risk of a variety of illnesses. Furthermore, catechins have been shown to suppress accumulation of visceral fat and so may be useful in controlling bodyweight and bodyshape (see, for example, T. Nagao et al., “Tea Catechins Suppress Accumulation of Body Fat in Humans”, J. Oleo. Sci., 2001, 50(9), pp.717-728). These studies, along with the increasing complexity of the consumer's palate have led to growth in the consumption of green tea, even in markets (such as the USA and Western Europe) where there is no tradition of green tea consumption.

Although, some of the health benefits of tea may be apparent at consumption rates as low as three cups per day (see, for example, U. Peters et al., “Does tea affect cardiovascular disease? A meta-analysis.”, American Journal of Epidemiology, 2001, 154, pp.495-503), many individuals do not even achieve this modest consumption rate on a long term basis.

There have been several attempts to provide beverages with enhanced levels of catechins.

European patent EP 0 762 836 B discloses a beverage whereby cellular hydration and drinkability are enhanced by the combination of green tea solids with selected levels and types of electrolytes and carbohydrates. Unfortunately, however, the presence of significant levels of carbohydrates in the beverage may compromise certain benefits delivered by the catechins. In particular, the high energy contribution of sugars would tend to counteract any benefits of the catechins in terms of controlling bodyweight and/or bodyshape.

European patent application EP 1 297 749 A discloses a beverage having a concentrated or purified tea extract incorporated therein, containing catechins from 0.092 to 0.5 wt. % and quinic acid in a specific weight ratio with respect to the catechins. The beverage is said to have an improved taste and does not cause an unpleasant aftertaste which will otherwise remain after bitterness or astringency peculiar to catechins is alleviated by the addition of a sweetener. However, the beverages disclosed therein require the addition of quinic acid which itself may bring unwanted taste to the beverage. Furthermore, the beverages are still unpleasantly bitter, especially for consumers used to tea-based beverages having relatively low levels of catechins, such as black tea.

Thus we have recognised that there is a need to provide a beverage which delivers the benefits of high levels of catechins, especially in respect of bodyweight and/or shape management, which beverage has improved taste, especially in respect of bitterness.

We have found that such a need may be met by providing a beverage having a specific composition.

Tests and Definitions

Beverage

As used herein the term “beverage” refers to a substantially aqueous drinkable composition suitable for human consumption. Preferably the beverage comprises at least 85% water by weight of the beverage, more preferably at least 90% and most preferably from 95 to 99.9%.

Tea Solids

As used herein, the term “tea solids” refers to dry material extractable from the leaves of the plant Camellia sinensis var. sinensis and/or Camellia sinensis var. assamica. The leaves may have been subjected to a so-called “fermentation” step wherein it is oxidised by certain endogenous enzymes that are released during the early stages of “black tea” manufacture. This oxidation may even be supplemented by the action of exogenous enzymes such as oxidases, laccases and peroxidases. Alternatively the leaves may have been partially fermented (“oolong” tea) or substantially unfermented (“green tea”).

Tea-based Beverage

As used herein, the term “tea-based beverage” refers to a beverage comprising at least 0.01% by weight tea solids by weight of the beverage. Preferably the tea-based beverage comprises from 0.04 to 3% tea solids, more preferably from 0.06 to 2%, most preferably from 0.1 to 1%.

Catechins

As used herein the term “catechins” is used as a generic term for catechin, gallocatechin, catechin gallate, gallocatechin gallate, epicatechin, epigallocatechin, epicatechin gallate, epigallocatechin gallate, and mixtures thereof.

Determination of Catechins and Caffeine in a Beverage

The amounts of catechins and caffeine in a beverage are determined simultaneously by reverse-phase HPLC as follows:

Sample Preparation

9 ml of the beverage are then taken and 1.12 ml of acetonitrile added, along with 1.12 ml of a solution of 2.5 mg/ml EDTA and 2.5 mg/ml ascorbic acid in distilled water. The resulting solution is then decanted into microcentrifuge tubes and centrifuged at a relative centrifugal force of 14000 g for 10 minutes.

HPLC Analysis Conditions

| | |
|---|---|
| Column: | Luna Phenyl hexyl 5μ, 250 × 4.60 mm |
| Flow rate: | 1 ml/min |
| Oven temperature: | 30° C. |
| Solvents: | A: 2% acetic acid in acetonitrile |
| | B: 2% acetic acid and 0.02 mg/ml EDTA in water |
| Injection volume: | 10 μl |

Gradient:

| Time | % Solvent A | % Solvent B | Step |
|---|---|---|---|
| 0 to 10 min | 5 | 95 | Isocratic |
| 10 to 40 min | 5-18 | 95-85 | Linear gradient |
| 40 to 50 min | 18 | 82 | Isocratic |
| 50 to 55 min | 50 | 50 | Wash |
| 55 to 75 min | 5 | 95 | Isocratic |

Quantification: Peak area relative to a calibration curve constructed daily. Calibration curve is constructed from caffeine and the concentration of catechins is calculated using the relative response factors of the individual catechins to caffeine (from the ISO catechin method - ISO/CD 14502-2). Individual caffeine standards (Sigma, Poole, Dorset, UK) are used as peak identification markers.

Sugars

As used herein the term “sugars” refers exclusively to digestible mono- and di-saccharides. This includes any sugars from tea solids, milk and fruit juice.

Total Energy Content

The total energy content (E) of a beverage is defined as the sum of the metabolizable energy of the ingredients present within 100 g of the beverage. It is calculated using Equation 1:

$$E = {\sum }_{j} {f}_{j} {c}_{j} , \qquad ( 1 )$$

wherein cⱼ is the mass of ingredient j in 100 g of the beverage and fⱼ is the calorie conversion factor for the ingredient j.

For digestible saccharides and proteins, fⱼ is typically 16.7 kJ (4.0 kcal) per g. For fats, fⱼ is 37.7 kJ (9.0 kcal) per g.

Calorie conversion factors are readily available from ingredient manufacturers. Alternatively, the factors may be determined from analysis and comparison of the composition and heats of combustion of dietary material with those of excreted material (see D. A. T. Southgate and J. V. G. A. Durnin, “Calorie conversion factors. An experimental reassessment of the factors used in the calculation of the energy value of human diets.”, British Journal of Nutrition, 1970, 24, pp. 517-535).

Non-nutritive Sweetener

Non-nutritive sweeteners consist of intense sweeteners, sugar alcohols and mixtures thereof. Intense sweeteners are those materials with a relative sweetness of at least 10, more preferably from 30 to 30000. The intense sweeteners include aspartame, saccharin, acesulfame K, alitame, thaumatin, cyclamate, glycyrrhizin, stevioside, neohesperidine, sucralose, monellin, neotame, mogroside V, MGGR, periandrin I-V, rebaudioside A, stevia extract and mixtures thereof. Sugar alcohols include HSH (hydrogenated starch hydrosylate—also known as polyglycitol), eythritol, arabitol, glycerol, xylitol, sorbitol, mannitol, lactitol, maltitol, isomalt, palatinit and mixtures thereof.

Sweetness

As defined herein, the relative sweetness, Rᵢ, refers to the sweetness of substance i relative to the sweetness of an equivalent weight of sucrose (i.e. sucrose has a relative sweetness of 1). This is a common means of expressing sweetness in the food industry. The relative sweetness of certain non-nutritive sweeteners is given in Table 1.

The sweetness in terms of sucrose, Sᵢ, of non-nutritive sweetener i present in an amount of mᵢ (percent by weight of the beverage), is the product of the relative sweetness and the amount, i.e. Sᵢ=Rᵢmᵢ. Thus, for a mixture of non-saccharide sweeteners, the sweetness in terms of sucrose (S) is defined by Equation 2:

$$S = {\sum }_{i} {S}_{i} = {\sum }_{i} {R}_{i} {m}_{i} . \qquad ( 2 )$$

TABLE 1

| Non-Saccharide Sweetener (i) | Relative sweetness (Rᵢ) |
|---|---|
| Aspartame | 200 |
| Saccharin | 400 |
| Acesulfame K | 200 |
| Alitame | 2,000 |
| Thaumatin | 2,000 |
| Cyclamate | 35 |
| Glycyrrhizin | 50 |
| Stevioside | 100 |
| Neohesperidine | 1,500 |
| Sucralose | 500 |
| Monellin | 2,000 |
| Neotame | 10,000 |
| Mogroside V | 350 |
| MGGR | 940 |
| Periandrin I-V | 150 |
| Rebaudioside A | 300 |
| Stevia extract | 30 |
| HSH | 0.7 |
| Erythritol | 0.7 |
| Arabitol | 0.5 |
| Glycerol | 0.8 |
| Xylitol | 1.0 |
| Sorbitol | 0.6 |
| Mannitol | 0.6 |
| Lactitol | 0.4 |
| Maltitol | 0.8 |
| Isomalt | 0.6 |
| Palatinit | 0.5 |

### SUMMARY OF THE INVENTION

In a first aspect, the present invention provides a beverage having a total energy content of less than 17 kJ per 100 g, wherein the beverage comprises catechins in amount (C) of from 0.04 to 0.4% by weight of the beverage, and non-nutritive sweetener in an amount satisfying the following condition:
34<(S/C)<115,
wherein S is the sweetness of the non-nutritive sweetener in terms of sucrose.

We have found that such beverages provide a well-balanced flavour in terms of sweetness and bitterness. It is preferred that the ratio (S/C) is less than 105, more preferably less than 95 and most preferably less than 85. Preferably also, the ratio (S/C) is at least 37, more preferably at least 40 and most preferably at least 45. To avoid the beverage becoming overly sweet, it is also preferred that the sweetness (S) is less than 10, more preferably less than 7 and most preferably less than 5% by weight of the beverage. Preferably also, the sweetness is at least 1, more preferably at least 2 and most preferably at least 3% by weight of the beverage.

The low energy content of the beverage allows for delivery of the health benefits of catechins without providing an unhealthy amount of calories. It is preferred that the total energy content is less than 5 kJ per 100 g, more preferably less than 2 kJ per 100 g and most preferably from 0.0001 to 1 kJ per 100 g.

Because the sweetness of the beverage is primarily provided by the non-nutritive sweetener it is preferred that the beverage comprises sugars in an amount of less than 0.25% by weight of the beverage, more preferably less than 0.1% and most preferably from 0.001 to 0.05%.

The concentration of non-nutritive sweetener will depend on the amount of catechins and the relative sweetness of the sweetener as set forth above. Typically, however, the beverage will comprise non-nutritive sweetener in an amount of between 0.00001 to 10% by weight of the beverage, more preferably from 0.001 to 1% and most preferably from 0.01 to 0.1%.

In order to minimise the energy contribution of the sweetener, it is preferred that the non-nutritive sweetener comprises intense sweetener. Preferably the non-nutritive sweetener comprises at least 50% intense sweetener by weight of the non-nutritive sweetener, more preferably at least 75% and most preferably from 90 to 100%. The most preferred intense sweeteners, owing to their well-rounded flavour, are sucralose, aspartame, acesulfame K, or a mixture thereof.

We have found that the said ratio of sweetener to catechins is particularly well-suited for delivering a tea-based beverage that has a refreshing tea flavour but is not too bitter. Thus it is preferred that the beverage is a tea-based beverage, preferably a green tea-based beverage.

From a standpoint of microbial stability and taste, it is preferred that the beverage has an acid pH. In particular, the pH (at 20° C.) may be from 2 to 5, more preferably from 2.5 to 4.5, most preferably from 3 to 4.

Suitable acidulants include organic acids such as citric, malic, tartaric, ascorbic, salts thereof and mixtures thereof. A particularly well-balanced flavour may be provided when the acid comprises malic acid and/or a salt thereof. Mixtures of citric acid (and/or its salt), malic acid (and/or its salt), and ascorbic acid (and/or its salt) also provide a good flavour. Typically, the concentration of the organic acid and/or salt in the beverage will be from 0.001 to 1%, more preferably from 0.01 to 0.5%.

The beverage preferably comprises flavour. Suitable flavours include natural and synthetic fruit flavours, and/or natural or synthetic herb flavours. Examples of fruit flavours include apple, peach, pear, lemon, lime, mandarin, grapefruit, cranberry, orange, strawberry, grape, kiwi, pineapple, passion fruit, mango, guava, raspberry and cherry. Examples of herb flavours include jasmine, chamomile, rose, mint, hawthorn, chrysanthemum, osmanthus, hibiscus, elderflower and verbena. Surprisingly, we have found that pineapple flavour (natural or synthetic) is particularly effective at masking the bitterness of catechins, therefore it is particularly preferred that the flavour comprises pineapple flavour. The pineapple flavour may be natural or synthetic. Preferably the pineapple flavour comprises 2-propenyl hexanoate. The amount of flavour is preferably between 0.001 and 0.5% by weight of the beverage, more preferably from 0.05 to 0.19%.

The beverage may additionally or alternatively comprise a weight management active. Suitable actives include biotin, pantothenic acid (vitamin B5), vitamin B6, niacin, magnesium, yerba mate extract, guarana extract, hydroxycitric acid and mixtures thereof.

In a most preferred embodiment, the beverage comprises caffeine in an amount of between 0.001 to 0.1% by weight of the beverage, more preferably from 0.005 to 0.05%. Caffeine is particularly preferred as it has been shown to assist in body management, especially in weight control and/or control of body shape.

The beverage of this invention may be used as a medicament or in the preparation of a medicament. In particular, the beverage may be used to provide any of the benefits associated with consumption of catechins such as treating and/or preventing cancer; and/or treating and/or preventing cardio-vascular disease. It is particularly preferred to use the beverage for controlling the bodyweight and/or shape of an individual. For example, the beverage may be used in a method of providing at least one of these benefits to an individual, the method comprising administering to the individual the beverage. Preferably the beverage is administered orally.

### DETAILED DESCRIPTION

The present invention will be further described with reference to the following example.

### EXAMPLE 1

Five beverage products were made having the formulations given in Table 2.

TABLE 2

| | Product 1 | Product 2 | Product 3 | Product 4 | Product 5 |
|---|---|---|---|---|---|
| Ingredient (g) | | | | | |
| Green tea powder* | 2.500 | 2.500 | 2.500 | 2.500 | 3.840 |
| Tri-sodium | 0.500 | 0.500 | 0.500 | 0.500 | 0.300 |
| citrate | | | | | |
| Citric acid | 1.000 | 1.000 | 1.000 | 1.000 | 0.900 |
| Malic acid | 0.500 | 0.500 | 0.500 | 0.500 | 0.300 |
| Ascorbic acid | 0.200 | 0.200 | 0.200 | 0.200 | 0.200 |
| Sucralose | 0.120 | 0.080 | 0.060 | 0.040 | 0.100 |
| Acesulfame K | 0.050 | 0.035 | 0.025 | 0.017 | 0.044 |
| Flavour** | 1.150 | 1.150 | 1.150 | 1.150 | 1.150 |
| Deionised Water | To 1000 | To 1000 | To 1000 | To 1000 | To 1000 |
| Properties | | | | | |
| C—Catechins | 0.07 | 0.07 | 0.07 | 0.07 | 0.11 |
| (wt. %) | | | | | |
| S—Sweetness | 7.0 | 4.7 | 3.5 | 2.3 | 5.9 |
| (wt. %) | | | | | |
| Caffeine (wt. %) | 0.013 | 0.013 | 0.013 | 0.013 | 0.021 |
| pH | 3.5 | 3.5 | 3.5 | 3.5 | 3.5 |

*Ceytea ™ from Premium Exports Ceylon Ltd (Sri Lanka).
**Elderflower and Virbena.

The products were made using the following protocol (all dissolution was performed at 20° C.):

1. Place half of the water in a glass beaker of the appropriate size.

2. Dissolve the green tea powder and tri sodium citrate together in a small amount of water and add to the glass beaker.

3. Dissolve flavours and sucralose in a small amount of water and add to the beaker.

3. Dissolve the acesulfame K in a small amount of water and add to the beaker.

4. Dissolve all the acids together (citric, malic and ascorbic) in a small amount of water and add to the beaker.

5. Add water to make up to the desired mass.

6. Fill the resulting solution into 330 ml cans and seal.

7. Pasteurise the cans at 70° C. for 10 minutes in a bench-top pasteuriser.

The canned products were stored at +5° C. for 1 week and then assessed by a taste panel. The results are shown in Table 3.

TABLE 3

| Product | (S/C) | Taste |
|---|---|---|
| 1 | 100 | Just acceptable - sweetness builds up in mouth and |
| | | tea flavour is weak. |
| 2 | 67 | Well-balanced flavour; not bitter and has |
| | | refreshing tea character. |
| 3 | 50 | Becoming unbalanced; slightly bitter. |
| 4 | 33 | Too bitter; refreshing but not pleasurable. |
| 5 | 54 | Well-balanced flavour; not bitter and has |
| | | refreshing tea character. |

1. A beverage having a total energy content of less than 17 kJ per 100 g, wherein the beverage comprises catechins in amount (C) of from 0.04 to 0.4% by weight of the beverage, characterised in that the beverage comprises non-nutritive sweetener in an amount satisfying the following condition:
34<(S/C)<115,
wherein S is the sweetness of the non-nutritive sweetener in terms of sucrose.

2. A beverage according to claim 1 wherein the sweetness (S) is less than 7% by weight of the beverage.

3. A beverage according to claim 1 wherein the total energy content is less than 5 kJ per 100 g, preferably less than 2 kJ per 100 g.

4. A beverage according to claim 1 wherein the beverage comprises sugars in an amount of less than 0.25% by weight of the beverage.

5. A beverage according to claim 1 wherein the beverage comprises non-nutritive sweetener in an amount of between 0.00001 to 10% by weight of the beverage.

6. A beverage according to claim 1 wherein the non-nutritive sweetener comprises intense sweetener.

7. A beverage according to claim 6 wherein the intense sweetener is sucralose, aspartame, acesulfame K, or a mixture thereof.

8. A beverage according to claim 1 wherein the beverage is a tea-based beverage.

9. A beverage according to claim 1 wherein the beverage has a pH of from 2 to 5.

10. A beverage according to claim 9 wherein the beverage has a pH of from 3 to 4.

11. A beverage according to claim 1 wherein the beverage comprises organic acid.

12. A beverage according to claim 11 wherein the organic acid comprises malic acid and/or a salt thereof.

13. A beverage according to claim 1 wherein the beverage comprises flavour.

14. A beverage according to claim 13 wherein the beverage comprises the flavour in an amount of between 0.001 and 0.5% by weight of the beverage.

15. A beverage according to claim 13 wherein the flavour comprises fruit flavour.

16. A beverage according to claim 15 wherein the fruit is pineapple.

17. A beverage according to claim 1 wherein the beverage comprises caffeine in an amount of between 0.001 to 0.1% by weight of the beverage.

18. A method of controlling the bodyweight and/or shape of an individual comprising the step of administering to the individual a beverage according to claim 1.

19. Use of a beverage according to claim 1 for the management of the bodyweight and/or shape of an individual.
